(19) United States
(12) Patent Application Publication
(10) Pub. No.: US 2026/0130685 A1
(43) Pub. Date: May 14, 2026

(54) DEVICES, SYSTEMS, AND METHODS FOR DELIVERING MEDICAL INSTRUMENTS WITHIN A PATIENT

(71) Applicant: Boston Scientific Scimed, Inc., Maple Grove, MN (US)

(72) Inventors: Jason N. Mehta, Austin, TX (US); Ryan Davis Gilkey, Austin, TX (US); Alexander R. Cohen, Austin, TX (US); John Mims, Austin, TX (US); Timothy Noah Foster-Frau, Austin, TX (US)

(73) Assignee: Boston Scientific Scimed, Inc., Maple Grove, MN (US)

(21) Appl. No.: 19/382,790

(22) Filed: Nov. 7, 2025

Related U.S. Application Data
(60) Provisional application No. 63/718,240, filed on Nov. 8, 2024.

(51) Int. Cl. A61B 17/32; A61B 17/00

(52) U.S. Cl. CPC A61B 17/320016; A61B 2017/0034

(57) ABSTRACT

A sidecar configured to be positioned alongside an elongate member configured to deliver a medical instrument, such as a medical scope. The sidecar is configured to deliver one or more medical instruments independently of a medical instrument delivered by the elongate member. The sidecar is coupled with the elongate member with the distal end of the sidecar proximal to the distal end of the elongate member. An auxiliary medical instrument delivered through a lumen of the sidecar is thus translatable as well as laterally movable with respect to a distal increased-flexibility section of the elongate member. A return-to-home feature may be provided to return the distal end of the auxiliary medical instrument from a position spaced apart from the elongate member to a position adjacent the elongate member.

### CROSS-REFERENCE TO RELATED APPLICATIONS

This application claims the benefit of priority under 35 U.S.C. § 119 to U.S. Provisional Application No. 63/718,240, filed Nov. 8, 2024, the entire disclosure of which is hereby incorporated by reference herein for all purposes.

### FIELD

The present disclosure relates generally to the field of medical devices which deliver medical instruments for performing a procedure within a patient's body. More particularly, the present disclosure relates to devices, systems, and methods which provide a second, auxiliary delivery device for a medical instrument along a first delivery device for a medical instrument. Each of the delivery devices may deliver a separate medical instrument and each is capable of motion independent of the motion of the other.

### BACKGROUND

Minimally-invasive procedures, such as transluminal, transcatheter, percutaneous, endoscopic, etc., procedures, have various benefits over open surgery. Whereas open surgery requires cutting open the patient's body to gain access into the patient's body and internal anatomical structures, minimally-invasive procedures access a target site within the patient via a natural orifice (or, in some instances, a small incision not considered to constitute an open-surgery cut). Such methods induce little to no bleeding (minimized to no surgical cutting), require minimal if any implements to hold open the access opening, reduce various risks typical of open surgery, reduce recovery times, and have various other benefits over open surgery. Despite the various benefits over open surgery, the limited available working space within a patient for performing minimally-invasive procedures presents various challenges. Often more than one medical instrument, tool, device, etc., is used during a given medical procedure. However, the lumen of a delivery device for such medical instruments, tools, devices, etc., often does not accommodate more than one medical instrument, tool, device, etc., to be extended therethrough. Therefore, multiple device exchanges may be required during the procedure. Moreover, the maneuverability of the instruments at the target site for the procedure may be limited by the delivery device through which the instrument extends. During Endo Luminal Surgery (ESL) procedures/endoscopic surgical procedures, there may be a need to add additional channels to a medical scope (e.g., an endoscope) to deploy multiple tools. However, maneuverability and control of the multiple channels and or devices may present a challenge, particularly if different movements and/or actions are to be performed by the different devices. For instance, during Endoscopic Submucosal Dissection (ESD), a physician may want to apply tissue traction to help visualize the separation being performed between the mucosa/submucosa and the muscle/muscularis. In this instance, there generally is a need to deploy a tool or device to apply traction, and then to make that tool or device independent of the movement directions of the endoscope (through which a tissue-separating device is extended). However, typical deployment of a traction device through the working channel of the endoscope limits the movement and operation of the traction device to being affected by, and not independent of, movements of the endoscope. As may be appreciated, the limited spaces in which minimally-invasive procedures typically are performed also impacts the maneuverability of various accessories, such as traction devices, within the body. Accordingly, there remains a need for improved devices, systems, and associated methods to deliver and use a medical device independently of another medical device, such as a medical scope, within a patient.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a system is disclosed for delivering medical instruments into a patient's body. In some aspects, the system includes a flexible tubular elongate member configured for insertion into the patient's body, and having a proximal end and a distal end, and a proximal portion proximal to a transition point between the proximal end and the distal end, and an increased-flexibility section distal to the transition point and more flexible than the proximal portion; and an auxiliary elongate member having an external elongate member with a proximal end and a distal end, and a distalmost end. In some aspects, the auxiliary elongate member is coupled to the primary elongate member with the distalmost end of the auxiliary elongate member adjacent or at the transition point along the primary elongate member; the flexible tubular elongate member defines a lumen configured to deliver a medical instrument therethrough and into the patient's body; and the auxiliary elongate member defines an auxiliary lumen between the proximal end and the distal end of the auxiliary elongate member and configured to deliver an additional medical instrument therethrough and into the patient's body exiting the auxiliary lumen proximal to the distal end of the primary elongate member.

In some aspects, the system further includes an axially translatable member extendable through the auxiliary lumen and having a distal end translatable between a position proximal to the distal end of the primary elongate member and a position distal to the distal end of the primary elongate member. In some aspects, the axially translatable member defines a lumen for delivery of an auxiliary medical instrument therethrough. In some aspects, the axially translatable member has a distal end configured to deliver an operable element of an auxiliary medical instrument into the patient's body. In some aspects, the system further includes an elongate flexible element coupling the flexible tubular elongate member and the axially translatable member, and translatable to adjust a distance between the flexible tubular elongate member and the axially translatable member. In some aspects, the system further includes a bracket configured for coupling of the elongate flexible element thereto and to be coupled with respect to the distal end of the flexible tubular elongate member. In some aspects, the elongate flexible element is translatable with respect to the bracket to adjust the distance between the flexible tubular elongate member and the auxiliary elongate member. In some aspects, the elongate flexible element has a distal end wrapped around the distal end of the axially translatable member to inhibit proximal translation of the distal end of the axially translatable member into the auxiliary lumen.

In some aspects, the flexible tubular elongate member is an insertion member of a medical scope.

In some aspects, the flexible tubular elongate member includes a control handle operable to move the increased-flexibility section of the flexible tubular elongate member with respect to the distal end of the auxiliary elongate member.

In accordance with various principles of the present disclosure, a system, for delivering medical instruments into a patient's body, includes a sheath configured to be inserted into a patient's body alongside an elongate member configured to deliver a medical instrument into a patient, the sheath having a proximal end, and a distal end, and defining a lumen therein extending between the proximal end and the distal end; an axially translatable member extendable through the lumen and having a distal end translatable between a position proximal to the distal end of the sheath and a position distal to the distal end of the sheath; and an elongate flexible element having a distal end fixedly coupled to the distal end of the axially translatable member, and a proximal end accessible by a medical professional from along the proximal end of the sheath, the elongate flexible element configured to be coupled with the elongate member to couple the distal end of the axially translatable member with the distal end of the elongate member.

In some aspects, the system further includes a bracket configured for coupling of the elongate flexible element thereto and to be coupled with respect to the distal end of the elongate member. In some aspects, the bracket defines a passage through which a portion of the elongate flexible element is translatable.

In some aspects, the axially translatable member has a distal end configured to deliver an operable element of an auxiliary medical instrument into the patient's body

In some aspects, the sheath is shaped along a length thereof to mate with at least a portion of the exterior of the elongate member.

In accordance with various principles of the present disclosure, a method of delivering and operating separate medical instruments includes delivering a first medical instrument with a primary elongate member having a proximal end and a distal end, and a proximal portion proximal to a transition point between the proximal end and the distal end, and an increased-flexibility section distal to the transition point and more flexible than the proximal portion; delivering a second medical instrument with an auxiliary elongate member extending along the primary elongate member with a distalmost end of the auxiliary elongate member at or along the transition point of the primary elongate member; and longitudinally translating the second medical instrument with respect to the distal end of the primary elongate member from a position proximal to the distal end of the primary elongate member.

In some aspects, the method further includes longitudinally translating the second medical instrument to position the distal end of the second medical instrument distal to the distal end of the primary elongate member. In some aspects, the method further includes manipulating the increased-flexibility section of the primary elongate member to move laterally away from the second medical instrument. In some aspects, the primary elongate member and the second medical instrument are coupled together via an elongate flexible element, the method further comprising axially translating the elongate flexible element with respect to at least one of the primary elongate member or the second medical instrument to allow the primary elongate member to move laterally away from the second medical instrument.

In some aspects, the method further includes manipulating the increased-flexibility section of the primary elongate member to move laterally away from the second medical instrument.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers with a ‘added at the end, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of a system, formed in accordance with aspects of the present disclosure, for delivering medical devices within a patient.

FIG. 2 illustrates a distal end of a system such as illustrated in FIG. 1 in use in a schematic illustration of a treatment site within a patient.

FIG. 3 illustrates a view similar to that of FIG. 2, but with the members of the system in different positions relative to one another.

FIG. 4 illustrates a distal end of a system similar to the system illustrated in FIG. 2, but with a different medical instrument, inserted directly through the auxiliary lumen of the system.

FIG. 5 illustrates an example of a use configuration of a distal end of a system such as illustrated in FIG. 2 with a different medical instrument extended through the auxiliary device.

FIG. 6 illustrates an elevational view of a distal end of another example of an embodiment of a system, formed in accordance with aspects of the present disclosure, for delivering medical devices within a patient.

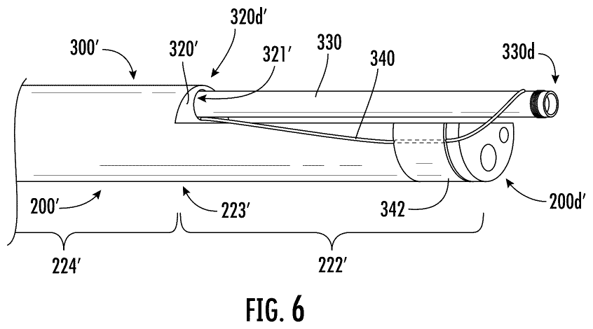

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

The present disclosure describes various medical devices and systems which may be delivered into a patient's body via minimally invasive techniques. More particularly, the present disclosure describes devices and systems, and associated methods, for delivering more than one medical device in the limited space in which minimally invasive procedures typically are performed. It will be appreciated that terms such as devices, instruments, tools, components, accessories, etc., may be used interchangeably herein without intent to limit unless otherwise indicated. The devices, systems, and methods of present disclosure further allow for independent movement of the medical devices which are delivered to a treatment site within the patient's body. It will be appreciated that terms such as treatment site, target site, target area, target tissue site, target tissue area, target area of tissue, target treatment area, treatment area, target treatment site, etc., may be used interchangeably herein, without intent to limit, to refer to an area or region of tissue at which a procedure is to be performed. The term target tissue (and other variations thereof, such as treatment tissue) is used herein to refer to the tissue with respect to which a procedure is to be performed or which is to be treated or otherwise operated on or affected by the devices and/or systems and/or methods disclosed herein. Thus, the treatment site may be understood as an area or region extending outwardly from or around or surrounding the target tissue (specific tissue in a target tissue area), such as the region a medical professional would consider a working area around the target tissue for performing a procedure with respect to the target tissue.

Minimally invasive procedures are becoming more desirable for removing or repairing target tissue within or in the area of a target site within a patient's body. In contrast with open surgery, minimally invasive surgery involves advancing a medical device into a patient's body through a natural orifice (or small incision, if necessary) and navigated the medical device within the body, such as through an internal body passage, to a target site. Typically, an elongate member is used in minimally-invasive medical procedures to deliver a medical device to a target site for use in performing a procedure. Typically, the elongate member is sufficiently flexible to navigate through curved and/or tortuous body passages, particularly if inserted transluminally. In some aspects, the elongate member is a shaft (substantially solid, or hollow and defining a lumen longitudinally therethrough) with a medical device at a distal end thereof. In some aspects, the elongate member is tubular, such as in the form of a catheter, sheath, tube, cannula, etc. (such terms being used interchangeably herein without intent to limit), and defines a lumen through which a medical device is distally advanced to a target site. A tubular elongate member may be used as a delivery device, introducer, etc. A tubular elongate member may or may not have a medical device mounted or otherwise coupled to a distal end thereof. In some aspects, it may be advantageous to deliver a medical device through a delivery device in the form of a tubular elongate member to protect or shield the passage through which the medical device is delivered (e.g., body passage or otherwise) against adverse interactions (e.g., catching, scratching, and/or otherwise) by the medical device delivered therethrough. Additional overtubes, sheaths, etc., may be provided as desired or as necessary to reduce friction or interference of the exterior of any elements with another element.

In addition to the above-described elongate members, various medical devices, such as medical scopes (e.g., endoscopes, arthroscopes, bronchoscopes, colonoscopes, cystoscopes, duodenoscopes, gastroscopes, hysteroscopes, laparoscopes, ureteroscopes, etc.), include an elongate member for insertion into a body passageway or cavity to enable a medical professional to deliver a medical device to a target site within a patient's body to perform minimally-invasive surgical procedures at the target site internal to the patient's body. Medical scopes typically have a proximal end (typically with a control handle) that remains external to the patient, and a distally extending flexible elongate member configured for insertion into the patient. The distally-extending flexible elongate member is generally tubular, and may be known as an insertion tube (and may referenced herein as such for the sake of convenience and without intent to limit). The distally-extending flexible elongate member of the medical scope is inserted into a body cavity or passage of the patient, and is navigated within the patient to an internal treatment site. In general, the distally-extending flexible elongate member of the medical scope has one or more working channels extending longitudinally therethrough through which a medical device may be advanced or retracted. The distally-extending flexible elongate member of the medical scope may also be equipped with one or more accessories such as, for example, a miniature viewing device (optical component, such as a camera), an illumination device (e.g., an LED or optical fiber), lumens for suction, lumens for inflation/irrigation mediums, etc. The distally-extending flexible elongate members of most medical scopes generally have a limited number of working channels, and the outer diameter of such devices generally is limited by the size of the body passage through which the device is to be navigated.

For purposes of the present disclosure, an elongate member with a medical device at a distal end thereof, or a tubular elongate member through which another medical device may be delivered to a target site, or the distally-extending flexible elongate member of a medical scope, or another form of an elongate member may be considered herein to be a delivery device, or, more generally an “elongate member” which delivers a medical instrument into a patient's body and with which systems, devices, and methods of the present disclosure may be used. For the sake of convenience, reference is made herein simply to an elongate member. It should be appreciated that such elongate member may have a medical device mounted or coupled to a distal end thereof (e.g., as an integral device), or may be a tubular elongate member with a medical device extended therethrough. The elongate member may be any suitable size, cross-sectional shape or area, and/or configuration permitting introduction and passage of devices or instruments to the distal end of the elongate member, such as for passage and introduction of medical instruments to a target tissue site. It will be appreciated that the elongate member need not define a lumen therethrough (need not be tubular for passage of a medical device therethrough), but, instead, may include a shaft with a medical device mounted thereon (e.g., on the distal end thereof). Typically, the elongate member has at least a distal portion which is elongate and flexible to be able to be navigated through passages within a patient's body which may be curved and/or tortuous, with a proximal portion extending proximally (out of the patient's body or coupled with a control handle outside the patient's body) to be controlled (maneuvered, or one or more functions thereof, such as camera, irrigation, suction, light, etc., actuated) by a medical professional.

In some aspects, an elongate member may not be sized to readily deliver more than one medical instrument (e.g., may have a lumen with a limited size). In accordance with various principles of the present disclosure, an auxiliary elongate member is provided in addition to an elongate member such as described above. For the sake of differentiating the elongate members, the first elongate member may be referenced as the first or primary elongate member, and the second elongate member may be referenced as an additional or auxiliary elongate member. The auxiliary elongate member optionally provides one or more channels, in addition to one or more channels which may be provided by the elongate member, to deliver one or more additional devices within a patient's body, or may provide one or more additional devices without an additional channel. In some aspects, the auxiliary elongate member may be considered a “sidecar” to the primary elongate member providing additional devices, features, functionalities, etc., to the primary elongate member.

In accordance with various principles of the present disclosure, the auxiliary elongate member allows for delivery of additional/auxiliary medical devices while a medical device is delivered by the primary elongate member. In some aspects, it is desirable for the movement directions and/or operation of the elongate members to be independent of each other, and/or to have different ranges of motion. In accordance with various principles of the present disclosure, at least a portion of the auxiliary elongate member is separable from the primary elongate member, and therefore may allow spatially independent operation of medical devices delivered by the auxiliary elongate member and medical devices delivered by the primary elongate member. For instance, an auxiliary elongate member formed in accordance with various principles of the present disclosure may be longitudinally and/or laterally movable with respect to the primary elongate member to allow independent longitudinal and/or lateral movement of medical instruments respectively delivered by the auxiliary elongate member and the primary elongate member. Additionally or alternatively, the auxiliary elongate member may be operably associated with the primary elongate member to allow selective movement of the auxiliary elongate member with (e.g., tracking, following, controlled by, etc.) movement of the primary elongate members. As may be appreciated, an auxiliary elongate member formed in accordance with various principles of the present disclosure thereby provides greater flexibility to the medical professional in the use of medical devices and the operation of such devices with respect to tissue at a target site. The medical instruments delivered by a system such as described herein may be axially and/or laterally separated from each other during use within a patient's body.

Various embodiments of elongate members auxiliary to and movable with a primary elongate member yet separable and independently movable from the primary elongate member, and associated systems and methods, will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

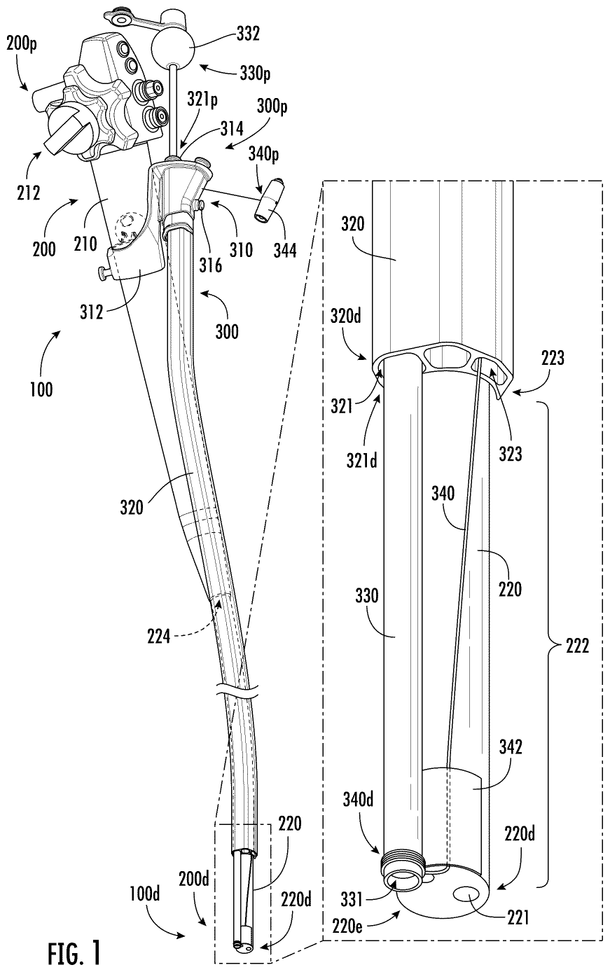

Turning now to the drawings, an example of an embodiment of a system 100 formed in accordance with various principles of the present disclosure for performing procedures within a patient's body is illustrated in FIG. 1. The system includes a primary elongate member 200, and an auxiliary elongate member 300 extending along the primary elongate member 200. In some aspects, the auxiliary elongate member 300 may be considered a “sidecar” to the primary elongate member 200, providing functionality and/or features, and/or delivering another medical instrument usable in conjunction with the primary elongate member 200. The example of an embodiment of a primary elongate member 200 is illustrated in the accompanying drawings as a medical scope. However, it will be appreciated that suitable configurations of a primary elongate member 200 other than a medical scope 200 may be utilized, the present disclosure not being limited in this regard. Reference is made herein to a medical scope 200 as the primary elongate member 200 solely for the sake of convenience, and without intent to limit.

The example of a medical scope 200 illustrated in FIG. 1 includes a proximal end 200p with a control handle 210, and a flexible tubular elongate member 220 (also known as a tubular shaft or insertion member of the medical scope) extending distally from the control handle 210 toward the distal end 200d of the medical scope 200. The flexible tubular elongate member 220 has a distal end 220d which is insertable into a patient and sufficiently flexible to be advanced to a target area within a patient's body in a manner known to those of ordinary skill in the art. The flexible tubular elongate member 220 defines one or more lumens or working channels 221 through which one or more medical tools may be extended and delivered to the target area. The distal end 220d of the flexible tubular elongate member 220 of the medical scope 200 typically has an increased-flexibility section 222 more flexible than the proximal portion 224 of the flexible tubular elongate member 220 proximal to the increased-flexibility section 222. The increased-flexibility section 222 may be considered as the portion of the flexible tubular elongate member 220 distal to a transition point 223, and the proximal portion 224 of the flexible tubular elongate member 220 may be considered as the portion of the flexible tubular elongate member 220 proximal to the transition point 223. The control handle 210 may include one or more control knobs 212 for controlling movement of the increased-flexibility section 222 of the flexible tubular elongate member 220, such as to direct the distalmost end 220e of the flexible tubular elongate member 220 toward a selected location (e.g., to direct a viewing element, light, working channel, etc., of the medical scope 200 to such location), in a manner known to those of ordinary skill in the art. For instance, small movements of the distalmost end 220e of the flexible tubular elongate member 220 may be needed to control movement of an instrument extended through the working channel 221 and out the distalmost end 220e of the flexible tubular elongate member 220.

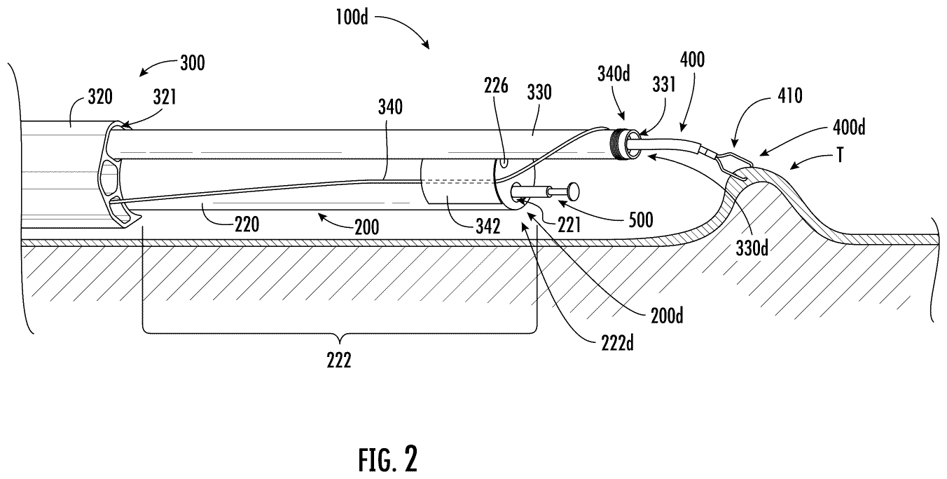

The example of an embodiment of an auxiliary elongate member 300 illustrated in FIG. 1 has a proximal end 300p with a control handle 310 to facilitate manipulation and control of components of the auxiliary elongate member 300 (as described in further detail below). The control handle 310 optionally includes a handle bracket 312 configured for operable engagement with the control handle 210 of the medical scope 200, such as to mount the control handle 310 with respect to the control handle 210 to facilitate access to both in close proximity. The auxiliary elongate member 300 further includes an external elongate member 320 configured to extend distally along the exterior of the flexible tubular elongate member 220 of the medical scope 200. The external elongate member 320 is sufficiently flexible to be navigated with the flexible tubular elongate member 220 into a patient's body. In some aspects, the external elongate member 320 is coupled to the flexible tubular elongate member 220 with an external coupler, such as a band, tie, strap, tape, etc. The external elongate member 320 may be in the form of a sheath configured to be inserted into a patient's body alongside the flexible tubular elongate member 220. In some aspects, at least a portion of the external elongate member 320 is shaped to complement or correspond to the exterior of the flexible tubular elongate member 220, such as to mate with or to receive at least a portion of the exterior of the flexible tubular elongate member 220 therealong and/or therein, such as may be appreciated with reference to the detail view in FIG. 1. The external elongate member 320 defines an auxiliary lumen 321 therethrough, which may be considered to define an additional or auxiliary lumen for use with the medical scope 200. For instance, an auxiliary medical instrument 400 in addition to a medical instrument 500 delivered by (e.g., through the working channel 221 of) the medical scope 200 may be delivered through the auxiliary lumen 321 of the external elongate member 320 to a treatment site, such as illustrated in FIG. 2, and described in further detail below. As may be appreciated, the provision of an auxiliary elongate member 300 in accordance with various principles of the present disclosure facilitates use of more than one tool at the same time during a procedure. For instance, a tissue cutting or separating tool may be extended through the working channel 221 of the flexible tubular elongate member 220 without the need to also accommodate a traction device, which may be delivered separately by the external elongate member 320. It will be appreciated that the external elongate member 320 may have more than one lumen extending from the proximal end 300p to the distal end 300d thereof, and may be configured to extend longitudinally and at least partially around the external periphery of the flexible tubular elongate member 220. It will be appreciated that although three lumens are illustrated as extending through the external elongate member 320, only one lumen, or only two lumens, or more than three lumens, or no lumens, may be provided (e.g., defined longitudinally through) the external elongate member 320. The auxiliary lumen 321 may be any of the lumens defined through the external elongate member 320.

In accordance with various principles of the present disclosure, the distal end 320d of the external elongate member 320 of the auxiliary elongate member 300 is operably coupled (e.g., mounted, attached, connected, etc.) to the flexible tubular elongate member 220 of the medical scope 200 at a location proximal to the distal end 220d of the flexible tubular elongate member 220. For instance, the distal end 320d of the external elongate member 320 may be operably coupled to the flexible tubular elongate member 220 at (substantially longitudinally aligned with) the transition point 223 along the flexible tubular elongate member 220. Such coupling allows a medical tool extended through the auxiliary lumen 321, and out the distal end 321d of the auxiliary lumen 321 and the distal end 320d of the external elongate member 320, to have a degree of freedom of movement independent of the movement of the increased-flexibility section 222 of the flexible tubular elongate member 220 of the medical scope 200. For instance, an elongate medical tool 400 may be extended into the proximal end 321p of the auxiliary lumen 321 of the external elongate member 320 (e.g., into a port 314 of the control handle 310 operably communicated with the auxiliary lumen 321) and out the distal end 321d of the auxiliary lumen 321 to extend distally out of the distal end 320d of the external elongate member 320. The elongate medical tool 400 may be longitudinally translatable distally toward, and optionally beyond, the distal end 220d of the flexible tubular elongate member 220, such as illustrated in FIG. 2. As such, the elongate medical tool 400 has a longitudinal range of movement independent of, and optionally greater than, a tool delivered by the medical scope 200 to a treatment site.

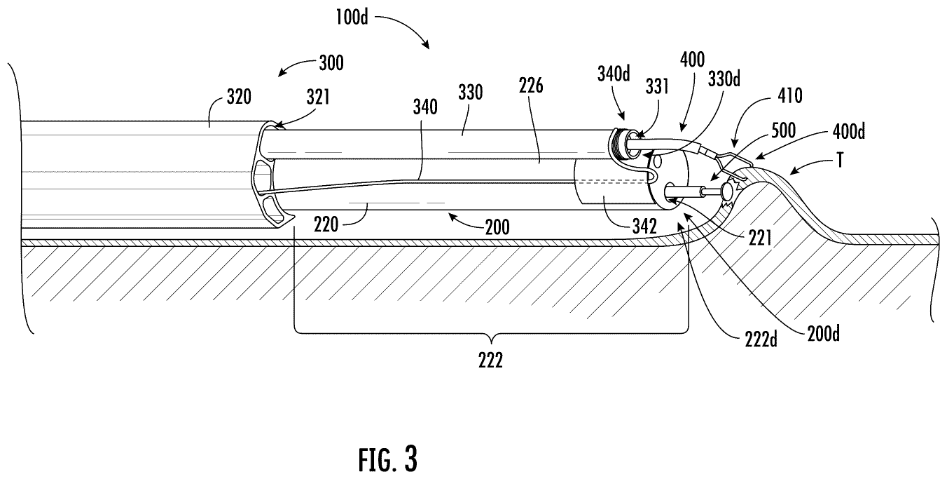

In some aspects, the auxiliary elongate member 300 further includes an axially translatable channel 330 extending from the proximal end 300p of the auxiliary elongate member 300 to the distal end 300d of the auxiliary elongate member 300, such as illustrated in FIG. 1. In some aspects, the axially translatable channel 330 may be formed of a rigid proximal segment (e.g., a metal hypotube), such as to enhance pushability, coupled with a more flexible distal segment (e.g., a flexible polymeric tube, formed of a material such as a thermoplastic elastomer), such as to facilitate flexing, particularly when alongside the increased-flexibility section 222 of the flexible tubular elongate member 220 of the medical scope 200. The axially translatable channel 330 defines a tool lumen 331 through which the elongate medical tool 400 may be longitudinally translatable, as may be appreciated with reference to FIG. 1, FIG. 2, and FIG. 3. The axially translatable channel 330 is longitudinally translatable through the auxiliary lumen 321 of the external elongate member 320 and relative to the medical scope 200. The axially translatable channel 330 may be extended distally beyond the distal end 321d of the auxiliary lumen 321 and the distal end 320d of the external elongate member 320 to extend along the increased-flexibility section 222 of the flexible tubular elongate member 220 of the medical scope 200, such as illustrated in FIG. 1, FIG. 2, and FIG. 3. In some aspects, the axially translatable channel 330 is longitudinally advanceable to extend the distal end 330d thereof distal to the distal end 222d of the increased-flexibility section 222 of the medical scope 200, such as illustrated in FIG. 2. In some aspects, the axially translatable channel 330 may be proximally retracted and/or the medical scope 200 (and the external elongate member 320) may be distally advanced relative to the position of the axially translatable channel 330, such as to distally advance a medical instrument 500 to perform a procedure with respect to tissue. In some aspects, the distal end 330d of the axially translatable channel 330 may be positioned proximal to the distal end 222d of the increased-flexibility section 222 of the medical scope 200, such as illustrated in FIG. 3. An operable element 410 (e.g., a grasper) of an auxiliary medical instrument 400 delivered by the axially translatable channel 330 may thus continue to be engaged with tissue (and optionally remain in a desired position/location) while a medical instrument 500 delivered by the medical scope 200 may be distally advanced with respect to tissue and the operable element 410. It will be appreciated that the medical scope 200 and the external elongate member 320 may be advanced further distally than illustrated in FIG. 3, with the distal end 330d of the axially translatable channel 330 not advancing the same distance, and thus becoming positioned increasingly proximally relative to the distal end 222d of the increased-flexibility section 222 of the medical scope 200 (and with the grasped tissue pulled further over the distal end 200d of the medical scope 200) than illustrated in FIG. 3.

The proximal end 330p of the axially translatable channel 330 may extend proximally to outside the patient's body for manual control by a medical professional. For instance, as illustrated in FIG. 1, the proximal end 330p of the axially translatable channel 330 may extend proximally out of the port 314 of the control handle 310 of the auxiliary elongate member 300. A grasping handle 332 may be provided on the proximal end 330p of the axially translatable channel 330 to facilitate grasping and/or longitudinal translation of the axially translatable channel 330. A proximal end of the elongate medical tool 400 may extend along or through the grasping handle 332 for manual control of the elongate medical tool 400 (e.g., to longitudinally translate the elongate medical tool 400 within the axially translatable channel 330). Any of a variety of grasping and/or control handles known to those of ordinary skill in the art may be provided along the proximal end of the elongate medical tool 400, the present disclosure not being limited in this regard. In some aspects, the external elongate member 320 may be considered to serve as a bearing surface for longitudinal translation of the axially translatable channel 330 (and the elongate medical tool 400) along the flexible tubular elongate member 220 of the medical scope 200.

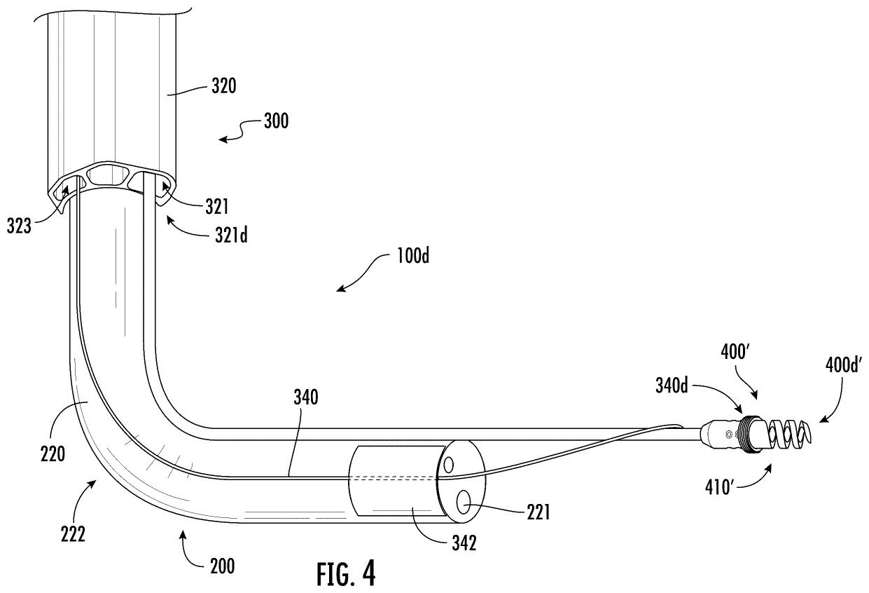

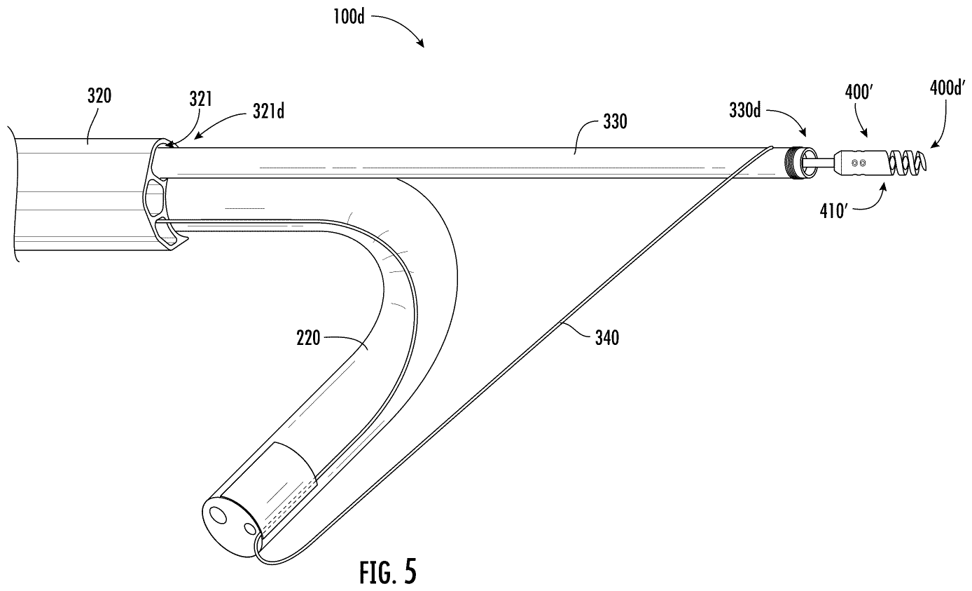

It will be appreciated that the elongate medical tool 400 may be delivered through the axially translatable channel 330 or independently (and without) an axially translatable channel 330. For instance, as illustrated in FIG. 4, an elongate medical tool 400′ may extend directly through the auxiliary lumen 321 defined through the external elongate member 320. Accordingly, reference may be made to movements and/or operation of the axially translatable channel 330 and the elongate medical tool 400, 400′ interchangeably and without intent to limit unless specified. Likewise, references to the movement and/or operation of an axially translatable channel 330 may be equally applicable to an elongate medical tool 400, 400′ which is provided directly through the auxiliary lumen 321 of the external elongate member 320 without an axially translatable channel 330. It will further be appreciated with reference to FIG. 2, FIG. 3, and FIG. 4 that the operable element (e.g., end effector or other element which may be used to perform a procedure) of the elongate medical tool 400, 400′ (e.g., extending from a distal end 400d, 400d′) need not be limited to a specific configuration. In the example of an embodiment illustrated in FIG. 2 and FIG. 3, the operable element 410 is a grasping end effector such as with a pair of movable jaws which can be moved from an open configuration to receive or acquire tissue therebetween, to a closed configuration grasping tissue therebetween. In the example of an embodiment illustrated in FIG. 4 and FIG. 5, the operable element 410′ of the elongate medical tool 400′ is a helical element, such as a helical tissue grasper which may be rotated into tissue and then longitudinally retracted to pull tissue proximally. The operable elements 410, 410′ may have other forms and functions, the present disclosure not being limited in this regard. It will be appreciated that although the operable elements 410, 410′ of the elongate medical tools 400, 400′ are not identical, the use of the elongate medical tools 400, 400′ in a system 100 formed in accordance with various principles of the present disclosure and described herein may be similar. As such, it will be appreciated that descriptions with reference to one of the elongate medical tools 400, 400′ may be equally applicable to the other of the elongate medical tools 400, 400′ unless specifically stated otherwise.

As may be appreciated, although the external elongate member 320 of the external elongate member 320 is maintained in place alongside the exterior of the flexible tubular elongate member 220 of the medical scope 200, once the axially translatable channel 330 extends distally out of the auxiliary lumen 321 of the external elongate member 320, the axially translatable channel 330 is no longer retained adjacent the flexible tubular elongate member 220 by the external elongate member 320. In accordance with various principles of the present disclosure, the axially translatable channel 330 (or the elongate medical tool 400, 400′ if an axially translatable channel 330 is not provided) may nonetheless be operably coupled with the increased-flexibility section 222 of the flexible tubular elongate member 220 via a return element 340. The axially translatable channel 330 and the elongate medical tool 400, 400′ may thereby be operably coupled with the increased-flexibility section 222 of the flexible tubular elongate member 220 to follow or track the movement (e.g., flexing, bending, etc.) of the increased-flexibility section 222, such as illustrated in FIGS. 2-5. In the illustrated examples of an embodiment, the return element 340 operably couples the distal end 330d of the axially translatable channel 330 (in FIG. 2 and FIG. 3) or the distal end 400d′ of the elongate medical tool 400′ (in FIG. 4 and FIG. 5) with the flexible tubular elongate member 220, such as with the distal end 222d of the increased-flexibility section 222. It will be appreciated that the location at which the return element 340 engages the axially translatable channel 330 (or the elongate medical tool 400′) and/or the flexible tubular elongate member 220 may be varied.

The return element 340 may be an elongate flexible element, such as, without limitation, a filament, a string, a cord, a wire, a tether, etc., coupled in any desired manner, such as, without intent to limit, wrapping, adhering, taping, integrating into a bracket or cap, etc., to the axially translatable channel 330 (or directly to the elongate medical tool 400′, such as illustrated in FIG. 4) and the flexible tubular elongate member 220. In the non-limiting example of an embodiment illustrated in FIG. 1 and FIG. 2, the return element 340 is wrapped around the distal end 330d of the axially translatable channel 330. Similarly, in the non-limiting example of an embodiment illustrated in FIG. 4 and FIG. 5, the return element 340 is wrapped around the distal end 400d′ of the elongate medical tool 400′. In some aspects, the return element 340 may be operably coupled with the distal end 330d of the axially translatable channel 330 in a configuration which serves as a hard stop for inhibiting or preventing further retraction of the axially translatable channel 330 proximally into the auxiliary lumen 321 of the external elongate member 320. For instance, the distal end 340d of the return element 340 may be wrapped around the distal end 330d of the axially translatable channel 330 to form an increased diameter which cannot be retracted into the auxiliary lumen 321.

In the non-limiting example of an embodiment illustrated in FIGS. 1-3, the return element 340 is coupled to an external coupler 342 positioned on the distal end 222d of the increased-flexibility section 222 of the flexible tubular elongate member 220. The external coupler 342 may be any structure known to those of ordinary skill in the art, such as a collar, bracket, cap, etc., capable of mounting along the exterior (e.g., partially or fully around the circumference) of the flexible tubular elongate member 220. In some aspects, the return element 340 slidably extends through a longitudinal channel passage 343 through or along the bracket 342 (such as for reasons as described below). It will be appreciated that other configurations of coupling the return element 340 with respect to the axially translatable channel 330 and/or the flexible tubular elongate member 220 are within the scope and spirit of the present disclosure. The return element 340 has a length allowing the distal end 340d of the return element 340 to be coupled along the distal end of the system 100, and allowing the proximal end 340p to be accessible from a proximal end of the system 100. It will be appreciated that the proximal end 340p of the return element 340 may be directly accessible, or may be coupled to another element which is accessible by a medical professional from a proximal end 100p of the system 100 (such as from outside the patient's body when the distal end 100d of the system 100 is within the patient's body).

In accordance with various principles of the present disclosure, in addition to the longitudinal movement an elongate medical tool 400, 400′ may have relative to the increased-flexibility section 222 of the medical scope 200, the elongate medical tool 400, 400′ and the increased-flexibility section 222 may be laterally movable with respect to each other, such as illustrated in FIG. 5. Because the elongate medical tool 400, 400′ is distally extendable beyond the distal end 320d of the external elongate member 320, the elongate medical tool 400, 400′ need not remain adjacent the flexible tubular elongate member 220 of the medical scope 200. For instance, if the external elongate member 320 is operably coupled to be adjacent the flexible tubular elongate member 220, the external elongate member 320 may constrain the portion of the axially translatable channel 330 and/or elongate medical tool 400, 400′ therein to be adjacent the flexible tubular elongate member 220. Once a portion of the axially translatable channel 330 and/or elongate medical tool 400, 400′ is extended beyond the distal end 320d of the external elongate member 320, the lateral position of such portion of the axially translatable channel 330 and/or elongate medical tool 400, 400′ is no longer constrained by the external elongate member 320 to be adjacent the flexible tubular elongate member 220. The elongate medical tool 400, 400′ thus need not remain laterally adjacent to the flexible tubular elongate member 220 of the medical scope 200. Moreover, the increased-flexibility section 222 of the flexible tubular elongate member 220 of the medical scope 200 may be moved, articulated, bent, flexed, etc. (such terms being usable interchangeably herein), independently of and without affecting the position, movement, configuration, etc., the axially translatable channel 330 or elongate medical tool 400, 400′.

In accordance with various principles of the present disclosure, the return element 340 may be operated to allow lateral separation of the axially translatable channel 330 from the flexible tubular elongate member 220, such as illustrated in FIG. 5, as well as return of the axially translatable channel 330 to a “home” position adjacent the flexible tubular elongate member 220, such as illustrated in FIGS. 1-4. In some aspects, such as noted above, the return element 340 is substantially fixedly coupled with the axially translatable channel 330 (in FIG. 1, FIG. 2, FIG. 3, and FIG. 5) or the elongate medical tool 400′ (in FIG. 4) and slidably coupled with respect to the flexible tubular elongate member 220. In the example of an embodiment illustrated in FIGS. 1-4, the return element 340 extends from the axially translatable channel 330 (in FIG. 1, FIG. 2, FIG. 3, and FIG. 5) or the elongate medical tool 400′ (in FIG. 4) and to the external coupler 342 on the flexible tubular elongate member 220. The return element 340 extends slidably and longitudinally translatably through the passage 343 defined along the external coupler 342, and slidably and longitudinally translatably along the flexible tubular elongate member 220 and the external elongate member 320 to a proximal end 340p outside the patient's body for manual grasping and manipulation by a medical professional. As illustrated in the example of an embodiment of FIG. 1, the return element 340 may extend through a return element lumen 323 defined longitudinally through the external elongate member 320 and proximally to the control handle 310 of the auxiliary elongate member 300. However, other pathways for the return element 340 to extend along the flexible tubular elongate member 220 and the external elongate member 320 (e.g., along the exterior of the flexible tubular elongate member 220, through another lumen defined through the external elongate member 320, etc.) are within the scope of the present disclosure. A grasping handle 344 (e.g., an enlarged grasping element, a knob, a lever, etc.) may be provided along the proximal end 340p of the return element 340 to facilitate grasping and/or manipulation of the return element 340. Would this include something like a lever (like a duodenoscope elevator), knob (to twist the string up)?

In some aspects, the proximal end 340p of the return element 340 may be operably coupled with the control handle 310 to maintain a desired length of the return element 340 extending between the elongate medical tool 400, 400′ and the flexible tubular elongate member 220 of the medical scope 200, thereby setting a distance (e.g., maximum distance) between the elongate medical tool 400, 400′ and the flexible tubular elongate member 220. In the example of an embodiment illustrated in FIG. 1, the control handle 310 may include a post 316 about which a proximal portion of the return element 340 may be wrapped to restrain the return element 340 against longitudinal movement. However, other structures may be used to retain the proximal end 340p of the return element 340 with respect to the control handle 310, such as a knob (e.g., ratcheting knob), a lever (e.g., a locking lever), a slider (e.g., a lockable slider), etc., such as in a manner known to those of ordinary skill in the art, the present disclosure not being limited in this regard. It will be appreciated that restraining the proximal end 340p of the return element 340 from moving maintains a desired length of the return element 340 and corresponding distance between the elongate medical tool 400, 400′ and the flexible tubular elongate member 220.

As may be appreciated, because the distal end 321d of the auxiliary lumen 321 of the external elongate member 320 ends proximal to the distal end 221d of the working channel 221 of the flexible tubular elongate member 220, the longitudinal portion of the axially translatable channel 330 extending distally out of the auxiliary lumen 321 is not constrained by the external elongate member 320 to be laterally adjacent the flexible tubular elongate member 220. If the axially translatable channel 330 is maintained adjacent the flexible tubular elongate member 220 by a return element 340, such as described above (and as illustrated in FIGS. 1-4), the return element 340 may be adjusted to allow the increased-flexibility section 222 to be laterally separated from and manipulated independently of the elongate medical tool 400, 400′. For instance, the proximal end 340p of the return element 340 may be released to allow slackening and/or relaxing and/or release of a length of the return element 340 between the elongate medical tool 400, 400′ and the flexible tubular elongate member 220 of the medical scope 200, such as illustrated in FIG. 5. The increased-flexibility section 222 of the flexible tubular elongate member 220, and any medical instrument 500 delivered by the flexible tubular elongate member 220, may thereby be navigated independently of the elongate medical tool 400, 400′. For instance, an elongate medical tool 400, 400′ delivered by the axially translatable channel 330 may remain in a desired position with respect to a treatment site while the distal end 220d flexible tubular elongate member 220 is navigated as desired or necessary during a procedure. Any of the accessories delivered by the increased-flexibility section 222, such as the working channel 221, a viewing device, an illumination device, inflation/irrigation/suction lumens, etc., may be navigated independently of the elongate medical tool 400, 400′. The system 100 of the present disclosure thereby provides greater range of motion, and increased independent movement and use of more than one tool during a procedure than provided by prior art systems.

As may be appreciated, at some points during a procedure, it may be desirable for the elongate medical tool 400, 400′ to remain close to (adjacent, and optionally contacting) the increased-flexibility section 222 of the flexible tubular elongate member 220 of the medical scope 200. For instance, it may be desirable for the elongate medical tool 400, 400′ to be within view of a viewing device of the medical scope 200. In order to return the axially translatable channel 330 and/or the elongate medical tool 400, 400′ to a position adjacent the increased-flexibility section 222, a medical professional may manipulate the return element 340 (e.g., move the return element 340 proximally, such as by pulling the proximal end 340p of the return element 340 proximally) to return the axially translatable channel 330 to a position closer to the flexible tubular elongate member 220. The return element 340 thus provides a return-to-home function to the axially translatable channel 330. In some aspects, the system 100 may only provide controlled longitudinal movement of the axially translatable channel 330 and may not provide controlled lateral movement of the axially translatable channel 330 other than via the return element 340. It will be appreciated that such homing feature may be used more generally to adjust the distance between the distal end 330d of the axially translatable channel 330 and the distal end 220d of the flexible tubular elongate member 220 (such as to adjust the positions of the operable element 410, 410′ of the elongate medical tool 400, 410′ and the medical instrument 500), and is not limited to returning the distal end 330d of the axially translatable channel 330 to a position adjacent to (e.g., in contact with) the distal end 220d of the flexible tubular elongate member 220.

An example of a procedure in which a system 100 formed in accordance with various principles of the present disclosure may be used is illustrated schematically in FIG. 2 and FIG. 3. The procedure may be an Endoscopic Submucosal Dissection (ESD) procedure in which submucosal tissue is cut, and traction is applied to tissue above the cut to help visualize the separation being performed between the mucosa/submucosa and the muscle/muscularis. A medical instrument 500 in the form of a tissue cutting or separating tool is illustrated extending distally out of a working channel 221 of the flexible tubular elongate member 220 of the medical scope 200 of the system 100 to cut tissue at a treatment site T. The medical instrument 500 may be a knife, such as an electrocautery knife, or a blunt tissue separator, or any other tool capable of separating tissue layers, such as for dissection or resection. The operable element 410 of the elongate medical tool 400 is illustrated as grasping tissue and pulling the tissue proximally (e.g., to apply traction to the tissue). Because the elongate medical tool 400, 400′ is longitudinally translatable with respect to the flexible tubular elongate member 220 of the medical scope 200 (such as described above), the operable element 410, 410′ of the elongate medical tool 400, 400′ may pull the grasped tissue proximally from the treatment site T to a position proximal to the distal end 220d of the flexible tubular elongate member 220, such as illustrated in FIG. 3. A viewing device 226 at the distal end 220d of the flexible tubular elongate member 220 may thus remain in place to view cutting performed by the cutting device 500, with the desired magnitude and direction of traction being applied to the tissue by the elongate medical tool 400, 400′. The magnitude of the physical displacement of grasped tissue (along a greater longitudinal extent than previously achievable) achieved by a system 100 formed in accordance with various principles of the present disclosure assists with performing the procedure without as much repositioning of the endoscope and maneuvering of other tools as required with previous systems. For instance, the displacement achieved by the increased range of motion of an elongate medical tool 400, 400′ in the system 100 of the present disclosure ensures visible exposure of the muscle/submucosal interface (which, in other systems, is normally achieved with scope movement and/or tunneling). In some aspects, the traction applied by the elongate medical tool 400, 400′ to the grasped tissue may assist with tissue dissection by tensioning the tissue fibers to allow the tissue fibers to break more easily. The medical instrument 500 may be a blunt tissue separator or dissector, with the increased traction applied by the elongate medical tool 400, 400′ (such as by pulling the tissue proximal to the distal end 220d of the flexible tubular elongate member 220) increasing effectiveness of the blunt tissue separator/dissector. Additionally or alternatively, displacement and/or gentle stretching of the tissue by a system 100 formed in accordance with various principles of the present disclosure may help to expose blood vessels, etc., which can then be treated conservatively (in contrast with potentially breaking of vessels which are not exposed by prior systems, requiring the medical professional to search for and then treat the broken vessel).

As may be appreciated in view of the above, the position of the distal end 321d of the auxiliary lumen 321 of the external elongate member 320 proximal to the distal end 221d of the working channel 221 of the flexible tubular elongate member 220 allows for increased range of movement and use of an elongate medical tool 400, 400′ delivered through the auxiliary lumen 321. The elongate medical tool 400, 400′ is not limited to use at the distal end 221d of the working channel 221 of the flexible tubular elongate member 220 as in prior devices delivered external to a medical scope 200. Instead, the elongate medical tool 400, 400′ is usable from a position adjacent to the distal end 321d of the auxiliary lumen 321 and proximal to the distal end 220d of the flexible tubular elongate member 220, to a position along or even distal to the distal end 220d of the flexible tubular elongate member 220. As such, the elongate medical tool 400, 400′ has a longitudinal range of use which is greater than tools provided through the working channel 221 of the flexible tubular elongate member 220, as well as auxiliary tools provided alongside the exterior of flexible tubular elongate members in prior systems. Such increased range of use of an elongate medical tool 400, 400′ may be advantageous in various procedures. For instance, a greater longitudinal range of motion of a medical tool may be useful in a procedure in which tissue is to be retracted, while a viewing device of a medical scope is used to view the treatment site.

As may be further appreciated, the position of the distal end 320d of the external elongate member 320 of the auxiliary elongate member 300 proximal to the distal end 220d of the flexible tubular elongate member 220 of the medical scope 200 of a system 100 formed in accordance with various principles of the present disclosure results in a system with distal end having a reduced profile compared to other medical scopes with external auxiliary devices. More specifically, when the axially translatable channel 330 is retracted within the auxiliary lumen 321 of the external elongate member 320, the distal end 100d of the system 100 is effectively the distal end 220d of the flexible tubular elongate member 220. In other words, because the distal end 320d of the external elongate member 320 is operably coupled with the flexible tubular elongate member 220 at a location proximal to the distal end 200d of the medical scope 200, the outer profile (e.g., cross-sectional area) of the distal end 100d of the system 100 may be defined by only the distal end 200d of the medical scope 200 (e.g., the increased-flexibility section 222 of the flexible tubular elongate member 220), and not also the auxiliary elongate member 300. The distal end 100d of the system 100 is thus typically more flexible than other systems which provide auxiliary channels or devices along the exterior of a medical scope. Such configuration of a system 100 formed in accordance with various principles of the present disclosure may facilitate intubation, navigation within the patient, insertion into narrow spaces, etc. Once the system 100 is positioned at a desired/intended target, the axially translatable channel 330 may be extended distally to deliver an elongate medical tool 400, 400′ auxiliary to a tool delivered by the medical scope 200.

In some aspects, instead of the auxiliary elongate member 300 being formed separately from the medical scope 200, an auxiliary elongate member 300′ may be formed as a lateral extension of the medical scope 200′, such as illustrated in FIG. 5. More particularly, the auxiliary elongate member 300′ includes an external elongate member 320′ which defines an auxiliary lumen 321′ extending generally parallel to the working channel 221′ of the medical scope 200′ but along an exterior wall which ends spaced proximally from the distal end 200d′ of the medical scope 200′. In some aspects, the distal end 320d′ of the external elongate member 320′ is positioned proximal to or at or adjacent a transition point 223′ between an increased-flexibility section 222′ and a proximal portion 224′ of the flexible tubular elongate member 220′. Such configuration of a medical scope 200′ formed with an auxiliary elongate member 300′ provides a system 100′ with a distal low profile configuration with benefits similar to those provided by proximal spacing of the distal end 300d of a separately formed auxiliary elongate member 300 from the distal end 200d of medical scope 200 as described above. An axially translatable channel 330 may be distally extended out of the distal end 321d′ of the auxiliary lumen 321′ once the system 100 has been navigated to a desired/intended target in a manner as described above with reference to the example of an embodiment of a system 100 illustrated in FIGS. 1-5. The distal end 330d of the axially translatable channel 330 may be operably coupled with the distal end 222d′ of the increased-flexibility section 222′ with a return element 340 similar to the above-described return element 340, reference therefore being made to the above descriptions for the sake of brevity and without intent to limit. It will be appreciated that the configuration of the example of an embodiment of a system 100′ with a medical scope 200′ and auxiliary elongate member 300′ formed as a single unit may be particularly suitable for a disposable medical scope. In some aspects, disposable medical scopes may be beneficial, such as from an infection prevention standpoint, as a single-use device. Additionally or alternatively, a single unit configuration as illustrated in FIG. 5 may improve scope maneuverability over a combination of a medical scope and an additional device coupled to/mounted over the side of the medical scope (which may be more bulky and harder to maneuver than an integrated system such as the system 100′ illustrated in FIG. 5).

It will be appreciated that embodiments of the present disclosure may be configured for treating tissue within a patient's body, such as in the gastrointestinal system, the abdominal cavity, the digestive system, the urinary tract, the reproductive tract, the respiratory system, the cardiovascular system, the circulatory system, etc. Various further benefits of the various aspects, features, components, and structures of a device and system, and associated methods, such as described above, in addition to those discussed above, may be appreciated by those of ordinary skill in the art. In view of the above, it should be understood that the various embodiments illustrated in the figures have several separate and independent features, which each, at least alone, has unique benefits which are desirable for, yet not critical to, the presently disclosed devices, systems, and methods

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A system for delivering medical instruments into a patient's body, said system comprising:
a flexible tubular elongate member configured for insertion into the patient's body, and having a proximal end and a distal end, and a proximal portion proximal to a transition point between the proximal end and the distal end, and an increased-flexibility section distal to the transition point and more flexible than the proximal portion; and
an auxiliary elongate member having an external elongate member with a proximal end and a distal end, and a distalmost end;
wherein:
said auxiliary elongate member is coupled to said primary elongate member with the distalmost end of said auxiliary elongate member adjacent or at the transition point along said primary elongate member;
said flexible tubular elongate member defines a lumen configured to deliver a medical instrument therethrough and into the patient's body; and
said auxiliary elongate member defines an auxiliary lumen between the proximal end and the distal end of said auxiliary elongate member and configured to deliver an additional medical instrument therethrough and into the patient's body exiting the auxiliary lumen proximal to the distal end of said primary elongate member.

2. The system of claim 1, further comprising an axially translatable member extendable through the auxiliary lumen and having a distal end translatable between a position proximal to the distal end of said primary elongate member and a position distal to the distal end of said primary elongate member.

3. The system of claim 2, wherein said axially translatable member defines a lumen for delivery of an auxiliary medical instrument therethrough.

4. The system of claim 2, wherein said axially translatable member has a distal end configured to deliver an operable element of an auxiliary medical instrument into the patient's body.

5. The system of claim 2, further comprising an elongate flexible element coupling said flexible tubular elongate member and said axially translatable member, and translatable to adjust a distance between said flexible tubular elongate member and said axially translatable member.

6. The system of claim 5, further comprising a bracket configured for coupling of said elongate flexible element thereto and to be coupled with respect to the distal end of said flexible tubular elongate member.

7. The system of claim 6, wherein said elongate flexible element is translatable with respect to said bracket to adjust the distance between said flexible tubular elongate member and said auxiliary elongate member.

8. The system of claim 5, wherein said elongate flexible element has a distal end wrapped around the distal end of said axially translatable member to inhibit proximal translation of the distal end of said axially translatable member into the auxiliary lumen.

9. The system of claim 1, wherein said flexible tubular elongate member is an insertion member of a medical scope.

10. The system of claim 1, wherein said flexible tubular elongate member includes a control handle operable to move the increased-flexibility section of said flexible tubular elongate member with respect to the distal end of said auxiliary elongate member.

11. A system for delivering medical instruments into a patient's body, said system comprising:
a sheath configured to be inserted into a patient's body alongside an elongate member configured to deliver a medical instrument into a patient, said sheath having a proximal end, and a distal end, and defining a lumen therein extending between the proximal end and the distal end;
an axially translatable member extendable through the lumen and having a distal end translatable between a position proximal to the distal end of said sheath and a position distal to the distal end of said sheath; and
an elongate flexible element having a distal end fixedly coupled to the distal end of said axially translatable member, and a proximal end accessible by a medical professional from along the proximal end of said sheath, said elongate flexible element configured to be coupled with the elongate member to couple the distal end of said axially translatable member with the distal end of the elongate member.

12. The system of claim 11, further comprising a bracket configured for coupling of said elongate flexible element thereto and to be coupled with respect to the distal end of the elongate member.

13. The system of claim 12, wherein said bracket defines a passage through which a portion of said elongate flexible element is translatable.

14. The system of claim 11, wherein said axially translatable member has a distal end configured to deliver an operable element of an auxiliary medical instrument into the patient's body.

15. The system of claim 11, wherein said sheath is shaped along a length thereof to mate with at least a portion of the exterior of the elongate member.

16. A method of delivering and operating separate medical instruments, said method comprising:
delivering a first medical instrument with a primary elongate member having a proximal end and a distal end, and a proximal portion proximal to a transition point between the proximal end and the distal end, and an increased-flexibility section distal to the transition point and more flexible than the proximal portion;
delivering a second medical instrument with an auxiliary elongate member extending along the primary elongate member with a distalmost end of the auxiliary elongate member at or along the transition point of the primary elongate member; and
longitudinally translating the second medical instrument with respect to the distal end of the primary elongate member from a position proximal to the distal end of the primary elongate member.

17. The method of claim 16, further comprising longitudinally translating the second medical instrument to position the distal end of the second medical instrument distal to the distal end of the primary elongate member.

18. The method of claim 17, further comprising manipulating the increased-flexibility section of the primary elongate member to move laterally away from the second medical instrument.

19. The method of claim 18, wherein the primary elongate member and the second medical instrument are coupled together via an elongate flexible element, said method further comprising axially translating the elongate flexible element with respect to at least one of the primary elongate member or the second medical instrument to allow the primary elongate member to move laterally away from the second medical instrument.

20. The method of claim 16, further comprising manipulating the increased-flexibility section of the primary elongate member to move laterally away from the second medical instrument.
